(19) United States
(12) Patent Application Publication
(10) Pub. No.: US 2006/0128676 A1
(43) Pub. Date: Jun. 15, 2006

(54) Compositions of nicotinic agonists and therapeutic agents and methods for treating or preventing diseases or pain

(75) Inventors: Steven L. Shafer, Mountain View, CA (US); Pamela Flood, Closter, NJ (US); Thomas E. Jenkins, La Honda, CA (US)

Correspondence Address: Richard F. Trecartin;Dorsey & Whitney LLP, Intellectual Property Department, 555 California Street, 10th Floor, San Francisco, CA, 94104-1513, US

(73) Assignee: Pharmacofore, Inc.

(21) Appl. No.: 11/181,369

(22) Filed: Jul. 13, 2005

Related U.S. Application Data
(60) Provisional application No. 60/587,369, filed on Jul. 13, 2004.
(60) Provisional application No. 60/587,397, filed on Jul. 13, 2004.
(60) Provisional application No. 60/587,382, filed on Jul. 13, 2004.
(60) Provisional application No. 60/587,349, filed on Jul. 13, 2004.
(60) Provisional application No. 60/587,474, filed on Jul. 13, 2004.
(60) Provisional application No. 60/587,476, filed on Jul. 13, 2004.
(60) Provisional application No. 60/587,377, filed on Jul. 13, 2004.
(60) Provisional application No. 60/587,387, filed on Jul. 13, 2004.
(60) Provisional application No. 60/587,413, filed on Jul. 13, 2004.
(60) Provisional application No. 60/587,379, filed on Jul. 13, 2004.
(60) Provisional application No. 60/587,374, filed on Jul. 13, 2004.
(60) Provisional application No. 60/587,375, filed on Jul. 13, 2004.
(60) Provisional application No. 60/587,475, filed on Jul. 13, 2004.
(60) Provisional application No. 60/587,386, filed on Jul. 13, 2004.
(60) Provisional application No. 60/587,376, filed on Jul. 13, 2004.
(60) Provisional application No. 60/587,396, filed on Jul. 13, 2004.
(60) Provisional application No. 60/587,477, filed on Jul. 13, 2004.

(51) Int. Cl. A61K 31/4439; A61K 31/60; A61K 31/415; A61K 31/405; A61K 31/192

(52) U.S. Cl. 514/159; 514/338; 514/343; 514/165; 514/406; 514/569; 514/570; 514/420

(57) ABSTRACT

Disclosed herein are compositions of nicotinic agonists, salts, hydrates, solvates or prodrugs thereof and therapeutic agents salts, hydrates, solvates or prodrugs thereof, pharmaceutical compositions thereof and methods of using these compositions and pharmaceutical compositions thereof to treat or prevent disease or pain in a subject. Also disclosed are methods of treating disease or pain or preventing disease or pain in a subject with nicotinic agonists, salts, hydrates, solvates or prodrugs thereof and therapeutic agents, salts, hydrates, solvates or prodrugs thereof.

This application claims priority under 35 U.S.C. § 119 (e) from U.S. Provisional Application Ser. Nos. 60/587,369, 60/587,397, 60/587,382, 60/587,349, 60/587,474, 60/587,476, 60/587,377, 60/587,387, 60/587,413, 60/574,378, 60/587,379, 60/587,374, 60/587,375, 60/587,475, 60/587,386, 60/587,376, 60/587,396 and 60/587,477 filed on Jul. 13, 2004 which are herein incorporated by reference in their entirety.

### 1. TECHNICAL FIELD

The methods and compositions disclosed herein relate generally to treating or preventing disease or pain in a subject. More specifically, disclosed herein are compositions of nicotinic agonists, salts, hydrates, solvates or prodrugs thereof and therapeutic agents salts, hydrates, solvates or prodrugs thereof, pharmaceutical compositions thereof and methods of using these compositions and pharmaceutical compositions thereof to treat or prevent disease or pain in a subject. Also disclosed are methods of treating disease or pain or preventing disease or pain in a subject with nicotinic agonists, salts, hydrates, solvates or prodrugs thereof and therapeutic agents, salts, hydrates, solvates or prodrugs thereof.

### 2. BACKGROUND

The efficacy of therapeutic agents in the treatment of disease or in the treatment/prevention of pain is often limited by intrinsic potency or toxicity. The intrinsic potency of a therapeutic agent can be limited by extent to which the therapeutic agent participates in a given mechanistic pathway and/or the extent to which the targeted mechanistic pathway is capable of treating or preventing a particular disease.

Toxicity may be either mechanism-based or non-mechanism-based. An example of mechanism-based toxicity is provided by sodium-channel antagonists (e.g., local anesthetics). When applied directly to the nerve, local anesthetics block nerve conduction via antagonism of sodium channels located on the nerve membrane resulting in anesthesia. However, once absorbed from the injection site, the local anesthetics travel through the systemic circulation to the heart where they can cause undesired cardiovascular effects via the antagonism of sodium channels. In this case, the same mechanism—antagonism of sodium channels—accounts for both the therapeutic and toxic effects of the therapeutic agent. In the case of non-mechanism based toxicity, the mechanisms of the desired therapeutic effects and the undesired toxic effects may due to unrelated mechanisms. An example of non-mechanism-based toxicity is observed when gylcopeptide antibiotics, designed to interfere with the mechanisms critical for the development of the bacterial cell wall, cause renal toxicity.

Accordingly, what is needed are methods for improving the utility of therapeutic agents via a means that can increase the desired therapeutic effects without causing or increasing toxicity.

### 3. SUMMARY

The present invention satisfies this and other needs by providing compositions of nicotinic agonists, salts, hydrates, solvates or prodrugs thereof and therapeutic agents salts, hydrates, solvates or prodrugs thereof, pharmaceutical compositions thereof and methods of using these compositions and pharmaceutical compositions thereof to treat or prevent disease or pain in a subject. Also disclosed are methods of treating disease or pain or preventing disease or pain in a subject with nicotinic agonists, salts, hydrates, solvates or prodrugs thereof and therapeutic agents, salts, hydrates, solvates or prodrugs thereof.

In one aspect, a composition comprising a therapeutically effective amount of a nicotinic agonist or salts, hydrates, solvates or prodrugs thereof and a therapeutically effective amount of an therapeutic agent is provided. In some embodiments, the therapeutic agent is selected from the group consisting of non-steroidal anti-inflammatory drugs, COX1 inhibitors, COX2 inhibitors, NMDA antagonists, substance P antagonists, selective serotonin re-uptake inhibitors, selective norepinephrene re-uptake inhibitors, vanilloid receptor 1 agonists, capsaicin receptor agonists, monoamine oxidase inhibitors, anesthetic agents, tricyclic antidepressants, atypical antidepressants, benzodiazepines, skeletal muscle relaxants, migraine therapeutic agents, anti-convulsants, anti-hypertensives, anti-arrythmics, antihistamines, steroids, methylxanthines, caffeine, calcium channel antagonists, beta blockers, alpha2 adrenergic agonists, non-benzodiazepine hypnotics and botulinum toxin, or salts, hydrates, solvates or prodrugs thereof. In other embodiments, the therapeutic agent is selected from the group consisting of non-steroidal anti-inflammatory drugs, COX1 inhibitors and COX2 inhibitors.

In another aspect, pharmaceutical compositions are provided. In some embodiments, a pharmaceutical composition comprising a therapeutically effective amount of a nicotinic agonist or salts, hydrates, solvates prodrugs or metabolites thereof and a therapeutically effective amount of an therapeutic agent or salts, hydrates, solvates, prodrugs or metabolites thereof and a pharmaceutically acceptable vehicle is provided.

In still another aspect, methods of treating or preventing disease or pain in a subject are provided. In some embodiments, these methods comprise administering to the subject in need of such treatment or prevention the compositions and pharmaceutical compositions, supra. In other embodiments, nicotinic agonists or salts, hydrates, solvates, prodrugs or metabolites thereof and therapeutic agents or salts, hydrates, solvates, prodrugs or metabolites thereof are administered to a subject where the therapeutic index of the combination of nicotinic agonists or salts, hydrates, solvates, prodrugs or metabolites thereof and therapeutic agent or salts, hydrates, solvates, prodrugs or metabolites thereof is substantially similar to or greater than the therapeutic index of the therapeutic agent or salts, hydrates, solvates, prodrugs or metabolites thereof or the therapeutic index of the nicotinic agonist or salts, hydrates, solvates, prodrugs or metabolites thereof. In still other embodiments, nicotinic agonists or salts, hydrates, solvates, prodrugs or metabolites thereof and therapeutic agents or salts, hydrates, solvates, prodrugs or metabolites thereof are administered to a subject where the relief to the subject is substantially similar to or greater than the therapeutic index of the therapeutic agent or salts, hydrates, solvates, prodrugs or metabolites thereof or the therapeutic index of the nicotinic agonist or salts, hydrates, solvates, prodrugs or metabolites thereof.

### 4. DETAILED DESCRIPTION

### 4.1 Definitions

“Metabolite” as used herein, refers to any substance produced by metabolism of either a therapeutic agent or a nicotinic agonist.

“Pharmaceutically acceptable vehicle” as used herein, refers to a diluent, adjuvant, excipient or carrier with which the nicotinic agonist and/or therapeutic agent disclosed herein are administered.

“Preventing” or “prevention” as used herein, refers, in some embodiments, to inhibiting pain or disease, either physically, (e.g., stabilization of a discernible symptom) or physiologically, (e.g., stabilization of a physical parameter) or both. In other embodiments, “preventing” or “prevention” refers to delaying the onset of pain or disease.

“Prodrug” as used herein, refers to a derivative of an therapeutic agent or a nicotinic agonist that requires a transformation within the body to release the active drug. Prodrugs are frequently, although not necessarily, pharmacologically inactive until converted to the parent drug. A hydroxyl containing drug may be converted, for example, to a sulfonate, ester or carbonate prodrug, which may be hydrolyzed in vivo to provide the hydroxyl compound. An amino containing drug may be converted, for example, to a carbamate, amide, enamine, imine, N-phosphonyl, N-phosphoryl or N-sulfenyl prodrug, which may be hydrolyzed in vivo to provide the amino compound. A carboxylic acid drug may be converted, for example, to an ester (including silyl esters and thioesters), amide or hydrazide prodrug, which be hydrolyzed in vivo to provide the carboxylic acid compound. Prodrugs other than those described, supra, are well known to the ordinarily skilled artisan and are within the scope of the present disclosure.

“Salt” as used herein, refers to a salt of an therapeutic agent or nicotinic agonist which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, t-butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Subject” as used herein, refers to a mammal, which includes, but is not limited to, domestic animals and humans. In some embodiments, the subject is a human female.

“Treating” or “treatment” as used herein, refers, in some embodiments, to ameliorating the disease or pain (i.e., arresting or reducing the development of disease or pain or at least one of the clinical symptoms thereof). In other embodiments “treating” or “treatment” refers to ameliorating at least one physical parameter of disease or pain, which may not be discernible by the subject.

“Therapeutically effective amount” as used herein, refers to the amount of a nicotinic agonist or therapeutic agent that, when administered to a subject for treating or preventing disease or pain, is sufficient to effect such treatment or prevention of disease or pain. The “therapeutically effective amount” will vary depending on the agonist, the disease and its severity and the age, weight, etc., of the subject to be treated.

“Therapeutically effective lifetime” as used herein, refers to the lifetime (i.e., the amount of time) of the nicotinic agonist or therapeutic agent where the agonist is effective in treating or preventing disease or pain. The “therapeutically effective lifetime” will vary depending on the agonist, the disease and its severity and the age, weight, etc., of the subject to be treated.

“Therapeutically index” as used herein, refers to the ratio of between the toxic dose and the therapeutic dose of a drug. In some embodiments, the term is used as a measure of the releative safety of the drug for treatment(s).

Reference will now be made in detail to various embodiments. It will be understood that the invention is not limited to these embodiments. To the contrary, it is intended to cover alternatives, modifications, and equivalents as may be included within the spirit and scope of the allowed claims.

### 4.2 Compositions of Nicotinic Agonists and Therapeutic Agents

Compositions comprised of nicotinic agonists, salts, hydrates, solvates, prodrugs or metabolites thereof and analgesic agents, salts, hydrates, solvates, prodrugs or metabolites thereof and pharmaceutical compositions thereof are described herein. While not wishing to be bound by theory, nicotinic agonists may reduce the dose of therapeutic agents required to treat and/or prevent disease or pain via either additive or synergistic effect. In some embodiments, the net toxicities of the nicotinic agonists and therapeutic agents may be offset when delivered as a combination. Thus, the therapeutic window for analgesia and/or treatment of disease may be increased by a composition comprising a nicotinic agonist and an therapeutic agent. Additive or synergistic effect may require co-release of the nicotinic agonist and therapeutic agent.

Generally, nicotinic agonists are compounds which activate the nicotinic receptor. Nicotinic agonists include, but are not limited to, nicotine, meta-nicotine, DMPP, DMAC, 3-2,4-dimethoxybenzylidine anabaseine (DMBX-anabaseine), choline, acetylcholine, cytisine, GTS-21, DMPP, DMAC, epibatidine (Quian et al., U.S. Pat. No. 6,077,846), ABT-418, ABT-594 (Decker et al., Curr Top Med Chem. 2004, 4:369-384; Michelmore et al., Naunyn Schmiedebergs Arch Pharmacol 2002, 366(3): 235-45), SIB-1508 and SIB-1558 (Jain, Curr Opin Investig Drugs 2004, 5(1): 76-81). In some embodiments, the nicotinic agonist is nicotine. In other embodiments, nicotine is the (+) antipode. In still other embodiments, nicotine is the (−) antipode. In still other embodiments, nicotine is a mixture of the (+) antipode and the (−) antipode. In still other embodiments, nicotinic agonists do not include meta-nicotine.

In some embodiments, the nicotinic agonist is a partial nicotinic agonist. In some embodiments, the nicotinic agonist is a partial nicotinic agonist at one or more nicotonic receptor subtypes. In other embodiments, the nicotinic agonist is a full nicotinic agonist at one or more nicotonic receptor subtypes. In still other embodiments, the the nicotinic agonist is a full nicotinic agonist at one or more nicotonic receptor subtypes and is a partial nicotinic agonist at one or more nicotonic receptor subtypes.

In still other embodiments, the nicotinic agonist is selective for a α7 nicotinic receptor. A α7 nicotinic receptor can comprise only α7 subunits or alternatively can consist of α7 subunit(s) in conjunction with subunit(s) of other nicotinic subtypes. Accordingly, in some embodiments, the nicotinic receptor is a homopentamer while in other embodiments, the nicotinic receptor is a heteropentamer.

In some embodiments, a nicotinic agonist is selective for a α7 nicotinic receptor if it activates the α7 nicotinic receptor more than two fold in comparison to any other nicotinic receptor. In other embodiments, a nicotinic agonist is selective for a α7 nicotinic receptor if it activates the α7 receptor more than five fold in comparison to any other nicotinic receptor. In still other embodiments, a nicotinic agonist is selective for a α7 nicotinic receptor if it activates the α7 receptor more than ten fold in comparison to any other nicotinic receptor. In still other embodiments, a nicotinic agonist is selective for a α7 nicotinic receptor if it activates the α7 receptor more than twenty fold in comparison to any other nicotinic receptor. In still other embodiments, a nicotinic agonist is selective for a α7 nicotinic receptor if it activates the α7 receptor more than fifty fold in comparison to any other nicotinic receptor.

Examples of selective α7 receptor agonists include, but are not limited to, ABT-418, cocaine methiodide, 3-2,4-dimethoxybenzylidine anabaseine (DMXB-A), 3-(4-hydroxybenzylidene)anabaseine, 3-(4-methoxybenzylidene)anabaseine, 3-(4-aminobenzylidene)anabaseine, 3-(4-hydroxy-2-methoxybenzylidene)anabaseine, 3-(4-methoxy-2-hydroxybenzylidene)anabaseine, trans-3-cinnamylidene anabaseine, trans-3-(2-methoxy-cinnamylidene)anabaseine and trans-3-(4-methoxycinnamylidene)anabaseine, N-[(3R)-1-azabicyclo[2.2.2]oct-3-yl]-4-(4-hydroxyphenoxy)benzamide, N-[(3R)-1-azabicyclo[2.2.2]oct-3-yl]-4-(4-acetamidophenoxy)benzamide, N-[(3R)-1-azabicyclo[2.2.2]oct-3-yl]-4-(phenylsulfanyl)benzamide, and N-[(3R)-1-azabicyclo[2.2.2]oct-3-yl]-4-(3-chlorophenylsulphonyl)benzamide, (1-aza-bicyclo[2.2.2]oct-3-yl) carbamic acid 1-(2-fluorophenyl)-ethyl ester and compounds disclosed in Tracey et al., International Publication No. 2004/0502365.

In some embodiments, the therapeutic agent is selected from the group consisting of a non-steroidal anti-inflammatory drugs, NMDA antagonists, substance P antagonists, selective serotonin re-uptake inhibitors, selective norepinephrene re-uptake inhibitors, vanilloid receptor 1 agonists, monoamine oxidase inhibitors, local anesthetics, tricyclic antidepressants, atypical antidepressants, benzodiazepines, skeletal muscle relaxants, migraine therapeutic agents, anticonvulsants, antiarrythmics, antihistamines, steroids, methylxanthines including caffeine, calcium channel antagonists, beta blockers, alpha2 adrenergic agonists, non-benzodiazepine hypnotics and botulinum toxin, or salts, hydrates, solvates or prodrugs thereof. In other embodiments, the therapeutic agent is selected from the group consisting of non-steroidal anti-inflammatory drugs, COX1 inhibitors and COX2 inhibitors.

Non-steroidal anti-inflammatory drugs include, but are not limited to, aspirin, biphenylacetic acid, acetaminophen, diflunisal, choline magnesium trisalicylate, indomethacin, ibuprofen, ketoprofen, fenoprofen, oxaprozin, bromfenac, felbinac, piroxicam, naproxen, naproxen sodium, diclofenac potassium, mefenamic acid, meloxicam, piroxicam, nabumetone, nimesulfide, etodolac, keterolac, tolmetin, lysine acetylsalicylate, suprofen, suldinac, celecoxib, valdecoxib, parecoxib, refecoxib, rofecoxib, etoricoxib and lumiracoxib.

NMDA antagonists include, but are not limited to, dextromethorphan, 2-piperdinol-1-alkanol derivatives (See U.S. Pat. No. 5,272,160) eliprodil and ifenprodil, and ketamine.

Substance P antagonists include, but are not limited to, aprepitant and capsaicin.

Selective serotonin re-uptake inhibitors include citalopram, clomipramine, escalitopram, fluoxetine, fluvoxamine, nefazodone, paroxetine, and sertraline.

Selective norepinephrene re-uptake inhibitors include, but are not limited to, venlafaxine, nefazodone, milnacipran, desipramine or duloxetine.

Vanilloid receptor 1 agonists include, but are not limited to, capsazepine, 6-iodonordihydrocapsaicin, SNC 162, 4′-Chloro-3-methoxycinnamanilide, and resiniferatoxin and capsaicin.

Monoamine oxidase inhibitors include, but are not limited to isocarboxazid, phenelzine and tranylcypromine.

Local anesthetics include, but are not limited to, mepivacaine, etidocaine, ropivacaine, chloroprocaine, bupivacaine, procaine, and tetracaine.

Tricyclic antidepressants include, but are not limited to, amitriptyline, doxepin, desipramine, imipramine, nortriptyline, protriptyline, and sibutramine.

Typical antidepressants include, but are not limited to, buspirone, trazodone, venlaxine, mirtazapine, nefazodone and bupropion.

Benzodiazepines include, but are not limited to, alprazolam, chlordiazepoxide, clonazepam, diazepam, estazolam, flurazepam, lorazepam, oxazepam, midazolam, quazepam, temazepam and triazolam.

Skeletal muscle relaxants include, but are not limited to, baclofen, cyclobenzaprine, carisoprodol, chlorzoxazone, flavoxate, metaxolone, methocarbamol, orphenadrine and tizanidine.

Migraine therapeutic agents include, but are not limited to, almotriptan, eletriptan, frovatriptan, naratriptan, rizatriptan, sumatriptan, and zolmatriptan.

Anticonvulsants include, but are not limited to, carbamazepine, clorazepate, ethosuximide, ethotoin, gabapentin, lamotrigine, levetiracetam, mephobarital, methsuximide, nafarelin, phenyloin, primidone, tiagabine, topiramate, trimethadione, valproate, divalproex, oxcarbazepine, zonisamide and pregabalin.

Antiarrythmics include, but are not limited to, disopyramine, dofetilide, flecainide, ibutilide, mexiletine, moricizine, procainamide, propafenone, quinidine, and tocainide.

Antihistamines include, but are not limited to, azelastine, brompheniramine, cetirizine, chlorpheniramine, clemastine, cyproheptadine, desloratadine, dexchlorpheniramine, diphenhydramine, fexofenadine and loratadine.

Steroids include, but are not limited to, prednisone, methylprednisolone, betamethasone and dexamethasone.

Methylxanthines include, but are not limited to, caffeine, aminophylline, theophylline and oxtriphylline.

Calcium channel antagonists include, but are not limited to, amlodipine, diltiazem, felodipine, isradipine, nicardipine, nifedipine, nimodipine, nisoldipine and verapamil.

Beta blockers include, but are not limited to, sotalol, timolol, esmolol, carteolol, carvedilol, nadolol, propranolol, betaxolol, penbutolol, metoprolol, labetalol, acebutolol, atenolol, metoprolol, labetalol, pindolol and bisoprolol.

Alpha2 adrenergic agonists include, but are not limted to, clonidine and dexmedetomidine.

Non-benzodiazepine hypnotics include, but are not limited to, zolpidem and zaleplom.

In some embodiments, the nicotinic agonist is nicotine and the non-steroidal anti-inflammatory drug is aspirin, biphenylacetic acid, acetaminophen, diflunisal, choline magnesium trisalicylate, indomethacin, ibuprofen, ketoprofen, fenoprofen, oxaprozin, bromfenac, felbinac, piroxicam, naproxen, naproxen sodium, diclofenac potassium, mefenamic acid, meloxicam, piroxicam, nabumetone, nimesulfide, etodolac, keterolac, tolmetin, lysine, acetylsalicylate, suprofen, suldinac, celecoxib, valdecoxib, parecoxib, refecoxib, rofecoxib, etoricoxib or lumiracoxib. In other embodiments, the nicotinic agonist is nicotine and the non-steroidal anti-inflammatory drug is a COX-2 enzyme inhibitor. In some of these embodiments, the COX-2 enzyme inhibitor is celecoxib, valdecoxib, parecoxib, refecoxib, rofecoxib, etoricoxib or lumiracoxib.

As the various names of nicotinic agonists and/or analgesic agents used herein represent only one of the possible tautomeric or conformational forms, it should be understood that any tautomers or conformational isomers of nicotinic agonists and/or therapeutic agents as well as mixtures of these various different isomeric forms are encompassed by the present disclosure.

The nicotinic agonists and/or therapeutic agents described herein also include isotopically labeled nicotinic agonists and/or therapeutic agents where one or more atoms have an atomic mass different from the atomic mass conventionally found in nature. Examples of isotopes that may be incorporated into the nicotinic agonists and/or therapeutic agents disclosed herein include, but are not limited to, ²H, ³H, ¹¹C, ¹³C, ¹⁴C, ¹⁵N, ¹⁸O, ¹⁷O, etc. Nicotinic agonists and/or therapeutic agents described herein may exist in unsolvated forms as well as solvated forms, including hydrated forms and as N-oxides. In general, nicotinic agonists and/or therapeutic agents may be hydrated, solvated or N-oxides. Certain nicotinic agonists and/or therapeutic agents may exist in multiple crystalline or amorphous forms. All physical forms are equivalent for the uses contemplated herein.

Also provided by the instant disclosure is an article of manufacture, which comprises a packaging material having therein, a nicotinic agonist and an therapeutic agent with a label indicating a use of the nicotinic agonist as an adjuvant for disease or pain.

### 4.3 Therapeutic Methods of Use

In general, the compositions disclosed herein may be used to treat and/or prevent the same disease(s) and/or conditions as the therapeutic agent, as well-known in the art (see, e.g., Physicians Desk Reference, 2000 54^{th} Edition and the Merck Index, 13^{th} Edition).

In some embodiments, a composition comprising a therapeutically effective amount of a nicotinic agonist or salts, hydrates, solvates, prodrugs or metabolites thereof and a therapeutically effective amount of an therapeutic agent or salts, hydrates, solvates, prodrugs or metabolites thereof or pharmaceutical compositions thereof is administered to a subject to treat and/or prevent disease or pain. Also disclosed herein are methods of treating disease or pain with a combination of nicotinic agonists salts, hydrates, solvates, prodrugs or metabolites thereof and therapeutic agents, salts, hydrates, solvates, prodrugs or metabolites thereof where the therapeutic index of the combination of nicotinic agonist or salts, hydrates, solvates, prodrugs or metabolites thereof and therapeutic agent, salts, hydrates, solvates, prodrugs or metabolites thereof is substantially similar to or greater than the therapeutic index of the therapeutic agent or salts, hydrates, solvates, prodrugs or metabolites thereof or the therapeutic index of the nicotinic agonist or salts, hydrates, solvates, prodrugs or metabolites thereof. Also disclosed herein are methods of treating disease or pain with a combination of nicotinic agonists salts, hydrates, solvates, prodrugs or metabolites thereof and therapeutic agents, salts, hydrates, solvates, prodrugs or metabolites thereof where the relief to the subject is substantially similar or greater than the relief provided by administration of the therapeutically effective amount of the therapeutic agent or salts, hydrates, solvates or prodrugs thereof or the therapeutically effective amount nicotinic agonist or salts, hydrates, solvates or prodrugs thereof.

The type of pain which may treated by methods disclosed herein include, but are not limited to, acute pain, chronic pain, neuropathic pain, acute traumatic pain, arthritic pain, osteoarthritic pain, rheumatoid arthritic pain, muscular skeletal pain, post-dental surgical pain, dental pain, myofascial pain, cancer pain, visceral pain, diabetic pain, muscular pain, post-herpetic neuralgic pain, chronic pelvic pain, endometriosis pain, pelvic inflammatory pain and child birth related pain. Acute pain includes, but is not limited to, acute traumatic pain or post-surgical pain. Chronic pain includes, but is not limited to, neuropathic pain, arthritic pain, osteoarthritic pain, rheumatoid arthritic pain, muscular skeletal pain, dental pain, myofascial pain, cancer pain, diabetic pain, visceral pain, muscular pain, post-herpetic neuralgic pain, chronic pelvic pain, endometriosis pain, pelvic inflammatory pain and back pain. In some embodiments, when the subject is a human female, chronic pain includes, in particular, chronic pelvic pain, endometriosis pain, pelvic inflammatory pain and child birth related pain.

In some embodiments, the nicotinic agonist and the therapeutic agent are concurrently administered to a subject to treat or prevent disease or pain. For example, the nicotinic agonist may be released over the therapeutically effective lifetime of the analgesic agent. In other embodiments, particularly where the nicotinic agonist and the therapeutic agent are not part of the same composition or pharmaceutical composition thereof, the nicotinic agonist and the therapeutic agent may be administered sequentially as well as concurrently.

Without wishing to be bound by theory, maintenance of therapeutically effective amounts of nicotinic agonist over the therapeutically effective lifetime of the therapeutic agent may be necessary for effective synergy or additivity. Since clearance of nicotinic agonists may be rapid in comparison to that of many therapeutic agents, maintenance of therapeutically effective amounts of nicotinic agonists may require frequent nicotinic agonist administration at regular dosing intervals if performed sequentially. Alternatively, maintenance of therapeutically effective amounts of nicotinic agonists may require sustained release or constant infusion of nicotinic agonist when the nicotinic agonist and the therapeutic agent are concurrently administered.

### 4.4 Pharmaceutical Compositions

The pharmaceutical compositions disclosed herein comprise a composition of a therapeutically effective amount of a nicotinic agonist or salts, hydrates, solvates, prodrugs or metabolites thereof and an therapeutic agent or salts, hydrates, solvates, prodrugs or metabolites thereof with a suitable amount of a pharmaceutically acceptable vehicle, so as to provide a form for proper administration to a subject. Also provided herein are pharmaceutical compositions comprising a therapeutically effective amount of a nicotinic agonist or salts, hydrates, solvates, prodrugs or metabolites thereof and a pharmaceutically acceptable vehicle and a pharmaceutical composition comprising a therapeutically effective amount of an therapeutic agent or salts, hydrates, solvates, prodrugs or metabolites thereof and a pharmaceutically acceptable vehicle.

Suitable pharmaceutical vehicles include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compositions and compounds disclosed herein into preparations which can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions or any other form suitable for use known to the skilled artisan. In some embodiments, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 19th Edition, 1995).

Pharmaceutical compositions for oral delivery may be in the form of tablets, lozenges, aqueous or oily suspensions, granules, powders, emulsions, capsules, syrups, slurries, suspensions or elixirs, for example. Orally administered compositions may contain one or more optional agents, for example, sweetening agents such as fructose, aspartame or saccharin, flavoring agents such as peppermint, oil of wintergreen, or cherry coloring agents and preserving agents, to provide a pharmaceutically palatable preparation. Moreover, when in tablet or pill form, the compositions may be coated to delay disintegration and absorption in the gastrointestinal tract, thereby providing a sustained action over an extended period of time. Oral compositions can include standard vehicles such as mannitol, lactose, starch, magnesium stearate, sodium saccharine, cellulose, magnesium carbonate, sucrose, sorbitol, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP), granulating agents, binding agents and disintegrating agents such as the cross-linked polyvinylpyrrolidone, agar, or alginic acid or a salt thereof such as sodium alginate etc.

In some embodiments, pharmaceutical compositions are in the form of lozenges or lollipops where dissolution and release of the active ingredients occurs in the oral cavity, generally through the oral mucosa. For these embodiments, buffering agents may also be used to provide an optimum environment for delivery of the agents or compositions. Additional components may include, for example, sweeteners, binders, diluents, disintegrating agents, lubricating agents, etc. In some embodiments, a nicotinic agonist rich coating may be applied to the exterior of the oral transmucosal (lozenge or lollipop) delivery system, accelerating the initial onset of nicotinic agonist effect. The independent rates of delivery with an oral transmucosal delivery system may be achieved by adjusting the relative concentrations of the nicotinic agonist and therapeutic agent and/or by using different polymers with the nicotinic agonist and therapeutic agent which independently control their release rates into the mouth.

In still other embodiments, the pharmaceutical composition is a dissolving sublingual tablet, where dissolution and release of the active ingredients occurs under the tongue, and the compositions and/or compounds disclosed herein are absorbed through the oral mucosa. In these embodiments, buffer agents may also be used to provide an optimum environment for delivery of each of the agents. Additional components may include, for example, sweeteners, binders, diluents, disintegrating agents, etc. Additionally, a readily dissolvable coating containing the nicotinic agonist may be applied to the exterior of the sublingual tablet, accelerating the initial onset of nicotinic agonist effect. The independent rates of delivery with a sublingual tablet may be achieved by using different polymers with the nicotinic agonist and the therapeutic agent, and thus independently controlling release rates of these agents into the mouth.

The methods that involve oral administration of compositions and/or compounds disclosed herein of can also be practiced with a number of different dosage forms, which provide sustained release.

In some embodiments, the dosage form is comprised of beads that on dissolution or diffusion release compositions and/or compounds disclosed herein over an extended period of hours, preferably, over a period of at least 6 hours, more preferably, over a period of at least 8 hours and even more preferably, over a period of at least 12 hours and most preferably, over a period of at least 24 hours. The beads may have a central composition or core comprising compositions and/or compounds disclosed herein and pharmaceutically acceptable vehicles, including optional lubricants, antioxidants and buffers. The beads may be medical preparations with a diameter of about 1 to about 2 mm. Individual beads may comprise doses of the compositions and/or compounds disclosed herein. The beads, in some embodiments, are formed of non-cross-linked materials to enhance their discharge from the gastrointestinal tract. The beads may be coated with a release rate-controlling polymer that gives a timed-release profile.

The time-release beads may be manufactured into a tablet for therapeutically effective administration. The beads can be made into matrix tablets by direct compression of a plurality of beads coated with, for example, an acrylic resin and blended with excipients such as hydroxypropylmethyl cellulose. The manufacture of beads has been disclosed in the art (Lu, Int. J. Pharm. 1994, 112, 117-124; Pharmaceutical Sciences by Remington, 14^{th} ed, pp 1626-1628 (1970); Fincher, J. Pharm. Sci. 1968, 57, 1825-1835; Benedikt, U.S. Pat. No. 4,083,949) as has the manufacture of tablets (Pharmaceutical Sciences, by Remington, 17^{th} Ed, Ch. 90, pp 1603-1625 (1985).

In other embodiments, an oral sustained release pump may be used (Langer, supra; Sefton, 1987, CRC Crit Ref Biomed. Eng. 14:201; Saudek et al., 1989, N. Engl. J. Med. 321:574).

In still other embodiments, polymeric materials can be used (See “Medical Applications of Controlled Release,” Langer and Wise (eds.), CRC Press., Boca Raton, Fla. (1974); “Controlled Drug Bioavailability,” Drug Product Design and Performance, Smolen and Ball (eds.), Wiley, New York (1984); Langer et al., 1983, J Macromol. Sci. Rev. Macromol Chem. 23:61; Levy et al., 1985, Science 228: 190; During et al., 1989, Ann. Neurol. 25:351; Howard et al., 1989, J. Neurosurg. 71:105). In some embodiments, polymeric materials are used for oral sustained release delivery. Such polymers include, for example, sodium carboxymethylcellulose, hydroxypropylcellulose, hydroxypropylmethylcellulose and hydroxyethylcellulose (most preferred, hydroxypropylmethylcellulose). Other cellulose ethers have been described (Alderman, Int. J. Pharm. Tech. &Prod. Mfr. 1984, 5(3) 1-9). Factors affecting drug release are well known to the skilled artisan and have been described in the art (Bamba et al., Int. J. Pharm. 1979, 2, 307).

In still other embodiments, enteric-coated preparations can be used for oral sustained release administration. Coating materials include, for example, polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In yet other embodiments, drug-releasing lipid matrices can be used for oral sustained release administration. For example, solid microparticles of compositions and/or compounds disclosed herein may be coated with a thin controlled release layer of a lipid (e.g., glyceryl behenate and/or glyceryl palmitostearate) as disclosed in Farah et al., U.S. Pat. No. 6,375,987 and Joachim et al., U.S. Pat. No. 6,379,700. The lipid-coated particles can optionally be compressed to form a tablet. Another controlled release lipid-based matrix material which is suitable for sustained release oral administration comprises polyglycolized glycerides as disclosed in Roussin et al., U.S. Pat. No. 6,171,615.

In yet other embodiments, waxes can be used for oral sustained release administration. Examples of suitable sustained releasing waxes are disclosed in Cain et al., U.S. Pat. No. 3,402,240 (carnauba wax, candedilla wax, esparto wax and ouricury wax); Shtohryn et al., U.S. Pat. No. 4,820,523 (hydrogenated vegetable oil, bees wax, caranuba wax, paraffin, candelillia, ozokerite and mixtures thereof); and Walters, U.S. Pat. No. 4,421,736 (mixture of paraffin and castor wax).

In still other embodiments, osmotic delivery systems are used for oral sustained release administration (Verma et al., Drug Dev. Ind. Pharm. 2000, 26:695-708). In some embodiments, OROS® systems made by Alza Corporation, Mountain View, Calif. are used for oral sustained release delivery devices (Theeuwes et al., U.S. Pat. No. 3,845,770; Theeuwes et al., U.S. Pat. No. 3,916,899).

In yet other embodiments, a controlled-release system can be placed in proximity of the target of the compositions and/or compounds disclosed herein thus requiring only a fraction of the systemic dose (See, e.g., Goodson, in “Medical Applications of Controlled Release,” supra, vol. 2, pp. 115-138 (1984)). Other controlled-release systems are discussed in Langer, 1990, Science 249:1527-1533 may also be used.

In still other embodiments, the dosage form comprises compositions and/or compounds disclosed herein coated on a polymer substrate. The polymer can be an erodible, or a nonerodible polymer. The coated substrate may be folded onto itself to provide a bilayer polymer drug dosage form. For example, compositions and/or compounds disclosed herein can be coated onto a polymer such as a polypeptide, collagen, gelatin, polyvinyl alcohol, polyorthoester, polyacetyl, or a polyorthocarbonate and the coated polymer folded onto itself to provide a bilaminated dosage form. In operation, the bioerodible dosage form erodes at a controlled rate to dispense the compositions and/or compounds over a sustained release period. Representative biodegradable polymers comprise a member selected from the group consisting of biodegradable poly(amides), poly (amino acids), poly(esters), poly(lactic acid), poly(glycolic acid), poly(carbohydrate), poly(orthoester), poly (orthocarbonate), poly(acetyl), poly(anhydrides), biodegradable poly(dihydropyrans), and poly(dioxinones) which are known in the art (Rosoff, Controlled Release of Drugs, Chap. 2, pp. 53-95 (1989); Heller et al., U.S. Pat. No. 3,811,444; Michaels, U.S. Pat. No. 3,962,414; Capozza, U.S. Pat. No. 4,066,747; Schmitt, U.S. Pat. No. 4,070,347; Choi et al., U.S. Pat. No. 4,079,038; Choi et al., U.S. Pat. No. 4,093,709).

In other embodiments, the dosage form comprises compositions and/or compounds disclosed herein loaded into a polymer that releases the drug(s) by diffusion through a polymer, or by flux through pores or by rupture of a polymer matrix. The drug delivery polymeric dosage form comprises a concentration of 10 mg to 2500 mg homogenously contained in or on a polymer. The dosage form comprises at least one exposed surface at the beginning of dose delivery. The non-exposed surface, when present, is coated with a pharmaceutically acceptable material impermeable to the passage of the drug(s). The dosage form may be manufactured by procedures known in the art. An example of providing a dosage form comprises blending a pharmaceutically acceptable carrier like polyethylene glycol, with a known dose of compositions and/or compounds disclosed herein at an elevated temperature, (e.g., 37° C.), and adding it to a silastic medical grade elastomer with a cross-linking agent, for example, octanoate, followed by casting in a mold. The step is repeated for each optional successive layer. The system is allowed to set for about 1 hour, to provide the dosage form. Representative polymers for manufacturing the dosage form comprise a member selected from the group consisting of olefin, and vinyl polymers, addition polymers, condensation polymers, carbohydrate polymers, and silicone polymers as represented by polyethylene, polypropylene, polyvinyl acetate, polymethylacrylate, polyisobutylmethacrylate, poly alginate, polyamide and polysilicone. The polymers and procedures for manufacturing them have been described in the art (Coleman et al., Polymers 1990, 31, 1187-1231; Roerdink et al., Drug Carrier Systems 1989, 9, 57-10; Leong et al., Adv. Drug Delivery Rev. 1987, 1, 199-233; Roffet al., Handbook of Common Polymers 1971, CRC Press; Chien et al., U.S. Pat. No. 3,992,518).

In other embodiments, the dosage from comprises a plurality of tiny pills. The tiny time-release pills provide a number of individual doses for providing various time doses for achieving a sustained-release drug delivery profile over an extended period of time up to 24 hours. The matrix comprises a hydrophilic polymer selected from the group consisting of a polysaccharide, agar, agarose, natural gum, alkali alginate including sodium alginate, carrageenan, fucoidan, furcellaran, laminaran, hypnea, gum arabic, gum ghatti, gum karaya, grum tragacanth, locust bean gum, pectin, amylopectin, gelatin, and a hydrophilic colloid. The hydrophilic matrix comprises a plurality of 4 to 50 tiny pills, each tiny pill comprise a dose population of from 10 ng, 0.5 mg, 1 mg, 1.2 mg, 1.4 mg, 1.6 mg, 5.0 mg, etc. The tiny pills comprise a release rate-controlling wall of 0.001 mm up to 10 mm thickness to provide for the timed release of drug(s). Representative wall forming materials include a triglyceryl ester selected from the group consisting of glyceryl tristearate, glyceryl monostearate, glyceryl dipalmitate, glyceryl laureate, glyceryl didecenoate and glyceryl tridenoate. Other wall forming materials comprise polyvinyl acetate, phthalate, methylcellulose phthalate and microporous olefins. Procedures for manufacturing tiny pills are disclosed in Urquhart et al., U.S. Pat. No. 4,434,153; Urquhart et al., U.S. Pat. No. 4,721,613; Theeuwes, U.S. Pat. No. 4,853,229; Barry, U.S. Pat. No. 2,996,431; Neville, U.S. Pat. No. 3,139,383; Mehta, U.S. Pat. No. 4,752,470.

In other embodiments, the dosage form comprises an osmotic dosage form, which comprises a semipermeable wall that surrounds a therapeutic composition comprising compositions and/or compounds disclosed herein. In use within a subject, the osmotic dosage form comprising a homogenous composition, imbibes fluid through the semipermeable wall into the dosage form in response to the concentration gradient across the semipermeable wall. The therapeutic composition in the dosage form develops osmotic pressure differential that causes the therapeutic composition to be administered through an exit from the dosage form over a prolonged period of time up to 24 hours (or even in some cases up to 30 hours) to provide controlled and sustained release. These delivery platforms can provide an essentially zero order delivery profile as opposed to the spiked profiles of immediate release formulations.

In other embodiments, the dosage form comprises another osmotic dosage form comprising a wall surrounding a compartment, the wall comprising a semipermeable polymeric composition permeable to the passage of fluid and substantially impermeable to the passage of compositions and/or compounds disclosed herein present in the compartment, a drug-containing layer composition in the compartment, a hydrogel push layer composition in the compartment comprising an osmotic formulation for imbibing and absorbing fluid for expanding in size for pushing the drug composition layer from the dosage form, and at least one passageway in the wall for releasing the composition. The method delivers the compositions and/or compounds disclosed herein by imbibing fluid through the semipermeable wall at a fluid imbibing rate determined by the permeability of the semipermeable wall and the osmotic pressure across the semipermeable wall causing the push layer to expand, thereby delivering the compositions and/or compounds disclosed herein from the dosage form through the exit passageway to a subject over a prolonged period of time (up to 24 or even 30 hours). The hydrogel layer composition may comprise 10 mg to 1000 mg of a hydrogel such as a member selected from the group consisting of a polyalkylene oxide of 1,000,000 to 8,000,000 weight-average molecular weight which are selected from the group consisting of a polyethylene oxide of 1,000,000 weight-average molecular weight, a polyethylene oxide of 2,000,000 molecular weight, a polyethylene oxide of 4,000,000 molecular weight, a polyethylene oxide of 5,000,000 molecular weight, a polyethylene oxide of 7,000,000 molecular weight and a polypropylene oxide of the 1,000,000 to 8,000,000 weight-average molecular weight; or 10 mg to 1000 mg of an alkali carboxymethylcellulose of 10,000 to 6,000,000 weight average molecular weight, such as sodium carboxymethylcellulose or potassium carboxymethylcellulose. The hydrogel expansion layer comprises 0.0 mg to 350 mg, in present manufacture; 0.1 mg to 250 mg of a hydroxyalkylcellulose of 7,500 to 4,500,00 weight-average molecular weight (e.g., hydroxymethylcellulose, hydroxyethylcellulose, hydroxypropylcellulose, hydroxybutylcellulose or hydroxypentylcellulose) in present manufacture; 1 mg to 50 mg of an osmagent selected from the group consisting of sodium chloride, potassium chloride, potassium acid phosphate, tartaric acid, citric acid, raffinose, magnesium sulfate, magnesium chloride, urea, inositol, sucrose, glucose and sorbitol; 0 to 5 mg of a colorant, such as ferric oxide; 0 mg to 30 mg, in a present manufacture, 0.1 mg to 30 mg of a hydroxypropylalkylcellulose of 9,000 to 225,000 average-number molecular weight, selected from the group consisting of hydroxypropylethylcellulose, hydroxypropypentylcellulose, hydroxypropylmethylcellulose, and hydropropylbutylcellulose; 0.00 to 1.5 mg of an antioxidant selected from the group consisting of ascorbic acid, butylated hydroxyanisole, butylated hydroxyquinone, butylhydroxyanisole, hydroxycoumarin, butylated hydroxytoluene, cephalm, ethyl gallate, propyl gallate, octyl gallate, lauryl gallate, propyl-hydroxybenzoate, trihydroxybutyrophenone, dimethylphenol, dibutylphenol, vitamin E, lecithin and ethanolamine; and 0.0 mg to 7 mg of a lubricant selected from the group consisting of calcium stearate, magnesium stearate, zinc stearate, magnesium oleate, calcium palmitate, sodium suberate, potassium laurate, salts of fatty acids, salts of alicyclic acids, salts of aromatic acids, stearic acid, oleic acid, palmitic acid, a mixture of a salt of a fatty, alicyclic or aromatic acid and a fatty, alicyclic or aromatic acid.

In the osmotic dosage forms, the semipermeable wall comprises a composition that is permeable to the passage of fluid and impermeable to the passage of compositions and/or compounds disclosed herein. The wall is non-toxic and comprises a polymer selected from the group consisting of a cellulose acylate, cellulose diacylate, cellulose triacylate, cellulose acetate, cellulose diacetate and cellulose triacetate. The wall comprises 75 wt % (weight percent) to 100 wt % of the cellulosic wall-forming polymer; or, the wall can comprise additionally 0.01 wt % to 80 wt % of polyethylene glycol, or 1 wt % to 25 wt % of a cellulose ether selected from the group consisting of hydroxypropylcellulose or a hydroxypropylalkylcellulose such as hydroxypropylmethylcellulose. The total weight percent of all components comprising the wall is equal to 100 wt %. The internal compartment comprises the drug-containing composition alone or in layered position with an expandable hydrogel composition. The expandable hydrogel composition in the compartment increases in dimension by imbibing the fluid through the semipermeable wall, causing the hydrogel to expand and occupy space in the compartment, whereby the drug composition is pushed from the dosage form. The therapeutic layer and the expandable layer act together during the operation of the dosage form for the release of compositions and/or compounds disclosed herein to a subject over time. The dosage form comprises a passageway in the wall that connects the exterior of the dosage form with the internal compartment. The osmotic powered dosage form can be made to deliver drug from the dosage form to the subject at a zero order rate of release over a period of up to about 24 hours.

The expression “passageway” as used herein comprises means and methods suitable for the metered release of the compositions and/or compounds disclosed herein from the compartment of the dosage form. The exit means comprises at least one passageway, including orifice, bore, aperture, pore, porous element, hollow fiber, capillary tube, channel, porous overlay, or porous element that provides for the osmotic controlled release of the compositions and/or compounds disclosed herein. The passageway includes a material that erodes or is leached from the wall in a fluid environment of use to produce at least one controlled-release dimensioned passageway. Representative materials suitable for forming a passageway, or a multiplicity of passageways comprise a leachable poly(glycolic) acid or poly(lactic) acid polymer in the wall, a gelatinous filament, poly(vinyl alcohol), leach-able polysaccharides, salts, and oxides. A pore passageway, or more than one pore passageway, can be formed by leaching a leachable compound, such as sorbitol, from the wall. The passageway possesses controlled-release dimensions, such as round, triangular, square and elliptical, for the metered release of compositions and/or drugs from the dosage form. The dosage form can be constructed with one or more passageways in spaced apart relationship on a single surface or on more than one surface of the wall. The expression “fluid environment” denotes an aqueous or biological fluid as in a human patient, including the gastrointestinal tract. Passageways and equipment for forming passageways are disclosed in Theeuwes et al., U.S. Pat. No. 3,845,770; Theeuwes et al., U.S. Pat. No. 3,916,899; Saunders et al., U.S. Pat. No. 4,063,064; Theeuwes et al., U.S. Pat. No. 4,088,864 and Ayer et al., U.S. Pat. No. 4,816,263. Passageways formed by leaching are disclosed in Ayer et al., U.S. Pat. No. 4,200,098 and Ayer et al., U.S. Pat. No. 4,285,987.

In order to decrease dosing frequency and augment the convenience to the subject and increase subject compliance, the sustained release oral dosage form (regardless of the specific form of the sustained release dosage form) preferably provides therapeutic concentrations of the compositions and/or compounds disclosed herein in the patient's blood over a period of at least about 6 hours, more preferably, over a period of at least about 8 hours, even preferably, over a period of at least about 12 hours and most preferably, over a period of at least 24 hours.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5 mM to about 50 mM), etc. Additionally, flavoring agents, preservatives, coloring agents, bile salts, acylcamitines and the like may be added.

Liquid drug formulations suitable for use with nebulizers and liquid spray devices and EHD aerosol devices will typically include compositions and/or compounds disclosed herein with a pharmaceutically acceptable carrier such as, for example, a liquid (e.g., alcohol, water, polyethylene glycol or a perfluorocarbon). Optionally, another material may be added to alter the aerosol properties of the solution or suspension of compositions and/or compounds disclosed herein. In some embodiments, this material is liquid such as an alcohol, glycol, polyglycol or a fatty acid. Other methods of formulating liquid drug solutions or suspensions suitable for use in aerosol devices are known to those of skill in the art (Biesalski, U.S. Pat. No. 5,112,598; Biesalski, U.S. Pat. No. 5,556,611)

For topical administration a compound of the invention may be formulated as solutions, gels, ointments, creams, suspensions, etc. as are well-known in the art.

For buccal administration, the compositions may take the form of tablets, lozenges, lollipops, etc. formulated in conventional manner.

Compositions and/or compounds disclosed herein may also be formulated in rectal or vaginal compositions such as suppositories or retention enemas, e.g., containing conventional suppository bases such as cocoa butter or other glycerides.

Systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include but are not limited to sodium channel blockers, antibiotics, N-acetyl cysteine, homocysteine and phospholipids.

For injection, compositions and/or compounds disclosed herein may be formulated in aqueous solutions, such as physiologically compatible buffers such as Hanks' solution, Ringer's solution, physiological saline buffer or in association with a surface-active agent (or wetting agent or surfactant) or in the form of an emulsion (as a water-in-oil or oil-in-water emulsion). Suitable surface-active agents include, in particular, non-ionic agents, such as polyoxyethylenesorbitans (e.g., Tween.™. 20, 40, 60, 80 or 85) and other sorbitans (e.g., Span.™. 20, 40, 60, 80 or 85). Compositions with a surface-active agent may comprise between 0.05 and 5% surface-active agent or between 0.1 and 2.5% surface-active agent. The solution may contain formulatory agents such as suspending, stabilizing and/or dispersing agents. Alternatively compositions and compounds may be in powder form for constitution with a suitable vehicle, e.g., sterile pyrogen-free water, before use.

Suitable emulsions may be prepared using commercially available fat emulsions. The combination (or single components) may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. In some embodiments, EDTA is added as a preservative.

In addition to the formulations described previously, compositions and/or compounds disclosed herein may also be formulated as a depot preparation. Such long acting formulations may be administered by implantation (for example subcutaneously or intramuscularly) or by intramuscular injection. Thus, for example, compositions and/or compounds disclosed herein may be formulated with suitable polymeric or hydrophobic materials (for example as an emulsion in an acceptable oil) or ion exchange resins, or as sparingly soluble derivatives, for example, as a sparingly soluble salt.

### 4.5 Therapeutic/Prophylactic Administration And Doses

When used to treat and/or prevent disease or pain the compositions of nicotinic agonists and therapeutic agents and/or pharmaceutical compositions thereof may be administered alone or in combination with other pharmaceutical agents including other compositions of nicotinic agonists and therapeutic agents. The nicotinic agonists, therapeutic agents, compositions of nicotinic agonists and therapeutic agents thereof may be administered or applied per se or as pharmaceutical compositions. The specific pharmaceutical composition depends on the desired mode of administration, as is well known to the skilled artisan.

Nicotinic agonists, therapeutic agents, compositions of nicotinic agonists and therapeutic agents and/or pharmaceutical compositions thereof may be administered to a subject by intravenous bolus injection, continuous intravenous infusion, oral tablet, oral capsule, oral solution, intramuscular injection, subcutaneous injection, transdermal absorption, buccal absorption, intranasal absorption, inhalation, sublingual, intracerebrally, intravaginallly, rectally, topically, particularly to the ears, nose, eyes, or skin or any other convenient method known to those of skill in the art. In some embodiments, nicotinic agonists, therapeutic agents, compositions of nicotinic agonists and therapeutic agents and/or pharmaceutical compositions thereof are delivered via sustained release dosage forms, including oral sustained release dosage forms. Administration can be systemic or local. Various delivery systems are known, (e.g., encapsulation in liposomes, microparticles, microcapsules, capsules, “patient controlled analgesia” drug delivery systems, etc.) that can be used to administer nicotinic agonists, therapeutic agents, compositions of nicotinic agonists and therapeutic agents and/or pharmaceutical compositions.

Nicotinic agonists, therapeutic agents, compositions of nicotinic agonists and therapeutic agents and/or pharmaceutical compositions may also be administered directly to the lung by inhalation. For administration by inhalation, the compositions and/or compounds disclosed herein may be conveniently delivered to the lung by a number of different devices. For example, a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas may be used to deliver the compositions and/or compounds disclosed herein.

Alternatively, a Dry Powder Inhaler (“DPI”) device may be used to administer the compositions and/or compounds disclosed herein (See, e.g., Raleigh et al., Proc. Amer. Assoc. Cancer Research Annual Meeting, 1999, 40, 397). DPI devices typically use a mechanism such as a burst of gas to create a cloud of dry powder inside a container, which may then be inhaled by the patient. A popular variation is the multiple dose DPI (“MDDPI”) system, which allows for the delivery of more than one therapeutic dose. For example, capsules and cartridges of gelatin for use in an inhaler or insufflator may be formulated containing a powder mix of the compositions and/or compounds disclosed herein and a suitable powder base such as lactose or starch for these systems.

Another type of device that may be used to deliver the compositions and/or compounds disclosed herein is a liquid spray device supplied, for example, by Aradigm Corporation, Hayward, Calif. Liquid spray systems use extremely small nozzle holes to aerosolize liquid drug formulations that may then be directly inhaled.

In some embodiments, a nebulizer device is used to deliver the compositions and/or compounds disclosed herein. Nebulizers create aerosols from liquid drug formulations by using, for example, ultrasonic energy to form fine particles that may be readily inhaled (e.g., Verschoyle et al., British J. Cancer, 1999, 80, Suppl. 2, 96; Armer et al., U.S. Pat. No. 5,954,047; van der Linden et al., U.S. Pat. No. 5,950,619; van der Linden et al., U.S. Pat. No. 5,970,974).

In still other embodiments, an electrohydrodynamic (“EHD”) aerosol device is used to deliver the compositions and/or compounds disclosed herein. EHD aerosol devices use electrical energy to aerosolize liquid drug solutions or suspensions (see e.g., Noakes et al., U.S. Pat. No. 4,765,539; Coffee, U.S. Pat. No. 4,962,885; Coffee, International Publication No. WO 94/12285; Coffee, International Publication No. WO 94/14543; Coffee, International Publication No. WO 95/26234; Coffee, International Publication No. WO 95/26235; Coffee, International Publication No. WO 95/32807). Other methods of intra-pulmonary delivery of a compound of the invention will be known to the skilled artisan and are within the scope of the present disclosure.

Transdermal devices can also be used to deliver the compositions and/or compounds disclosed herein. In some embodiments, the transdermal device is a matrix type transdermal device (Miller et al., International Publication No. WO 2004/041324). In other embodiments, the transdermal device is a multi-laminate transdermal device (Miller, United States Patent Application Publication No. 2005/0037059). In still other embodiments, the transdermal device will have one or more reservoirs with nicotinic agonist, salts, hydrates, solvates or prodrugs thereof either formulated separately, or in combination with an therapeutic agent, salts, hydrates, solvates or prodrugs thereof. In the embodiments in which separate reservoirs are used, suitable membranes may be used to adjust the delivery rates of the components such that one is delivered prior to or simultaneously with the other. In some embodiments, the therapeutic agents present in the transdermal patch reservoir and the nicotinic agonist is present in the device (e.g., patch) adhesive as well as the reservoir such that delivery of the nicotinic agonist begins immediately upon contact between the patch and the patient's skin.

In some embodiments, the device is intended for acute analgesia, for example post-operative analgesia. In these embodiments, the initial dose of the nicotinic agonist will be delivered very rapidly. The rapid absorption of the nicotinic agonist may be accomplished, in part, by the addition of the agonist to the device adhesive. The analgesic agent may also be added to the adhesive to increase the onset rate of analgesia. The rate of delivery may be controlled by rate limiting membranes separating the reservoir from the skin, or by the use of embedded polymers within the reservoir that control the rate of nicotinic agonist release into the reservoir. In some embodiments, the nicotinic agonist and the therapeutic agent are optionally mixed with separate polymers with distinct release characteristics in a single reservoir. In other embodiments, the nicotinic agonist and the analgesic agent are optionally mixed with separate polymers with distinct release characteristics in different reservoirs. The therapeutic agent and the nicotinic agonist may be present in the reservoir in quantities sufficient to provide 24 hours of analgesia following an acutely painful stimulation, such as an operation. As mentioned, supra, the nicotinic agonist may be included in the same reservoir as the analgesic agent or in a different reservoir.

In other embodiments the device is intended for chronic analgesia, for example, pain from cancer, debilitating arthritis, etc. In some embodiments, the device is changed every three days. The rate of delivery may be controlled by rate limiting membranes separating the reservoir from the skin, or by the use of embedded polymers within the reservoir that control the rate of nicotinic agonist release into the reservoir. In some embodiments, the nicotinic agonist and the therapeutic agent are optionally mixed with separate polymers with distinct release characteristics in a single reservoir. In other embodiments, the nicotinic agonist and the therapeutic agent are optionally mixed with separate polymers with distinct release characteristics in different reservoirs. As mentioned, supra, the nicotinic agonist may be included in the same reservoir as the analgesic agent or in a different reservoir. In some embodiments, the transdermal device provides sufficient nicotinic agonist and analgesic agent for 24-168 hours of sustained delivery. In some embodiments, the transdermal device provides sufficient nicotinic agonist and analgesic agent for 72 hours of sustained delivery.

In some embodiments, particularly, when a transdermal device is used to deliver nicotinic agonists and/or therapeutic agents to treat and/or prevent chronic pain, the active agents are delivered in three distinct phases. The first phase commences upon application of the device where the nicotinic agonist and therapeutic agent are initially absorbed from the device. In the second phase, the nicotinic agonist and therapeutic agent are delivered at a constant rate to maintain constant plasma nicotinic agonist and therapeutic agent concentrations. In the third phase, the device is removed, and the nicotinic agonist and therapeutic agent wash out of depots in the skin immediately below the patch, into the systemic circulation. The characteristics of this wash out are determined by the agents and by the skin and not by the device.

The rate of the nicotinic agonist and therapeutic agent delivery in the first phase, immediately following application of the device may be adjusted to compensate for declining rate of delivery of the nicotinic agonist and therapeutic agent during the third phase, when the active agents are washing out of the patch. In this manner, a newly applied device introduces the nicotinic agonist and therapeutic agent to the systemic circulation in a manner that approximately compensates for the decreasing delivery from the removed device. In this manner, the transdermal devices may be changed at regular intervals, as required for subjects with chronic pain, while still maintaining steady drug concentrations and consistent levels of analgesia.

The amount of nicotinic agonists, therapeutic agents, compositions of nicotinic agonists and therapeutic agents and/or pharmaceutical compositions thereof that will be effective in the treatment or prevention of disease or pain in a subject will depend on the specific nature of the condition and can be determined by standard clinical techniques known in the art. For example, nicotinic agonists, therapeutic agents, compositions of nicotinic agonists and therapeutic agents and/or pharmaceutical compositions thereof may be titrated to provide adequate analgesia to the subject. The amount of nicotinic agonists, therapeutic agents, compositions of nicotinic agonists and therapeutic agents and/or pharmaceutical compositions thereof administered will, of course, be dependent on, among other factors, the subject being treated, the weight of the subject, the severity of the affliction, the manner of administration and the judgment of the prescribing physician.

In some embodiments, a suitable dosage level for a nicotinic agonist is between about 1 mg to about 25 mg per day. In other embodiments a suitable dosage level for a nicotinic agonist is between about 5 mg to about 10 mg per day. The nicotinic agonist may be administered up to six times per day. In some embodiments, the nicotinic agonist is administered between 1 and four times daily.

Celecoxib can be administered at a dosage level up to conventional dosage levels for such analgesics but in some embodiments is administered at a reduced level in accord with present invention. In some embodiments, a suitable dosage level for celecoxib is between about 20 mg to about 1000 mg per day. In other embodiments, a suitable dosage level for celecoxib is between about 40 mg to about 400 mg per day. In still other embodiments, a suitable dosage level for celecoxib is between about 50 mg to about 300 mg per day. Celecoxib may be administered up to six times per day. In some embodiments, celecoxib is administered between 1 and four times daily.

When administered in combination, either as a single or as a separate pharmaceutical composition the nicotinic agonist and celecoxib are present in a ratio consistent with the desired effect. In some embodiments the ratio by weight of celecoxib to the nicotinic agonist is about 20:1. In other embodiments, the ratio by weight of celecoxib to the nicotinic agonist is between about 200:1 and about 2:1. In still other embodiments, the ratio by weight of celecoxib to the nicotinic agonist is between about 40:1 and about 10:1.

Indomethacin can be administered at a dosage level up to conventional dosage levels for such analgesics but in some embodiments is administered at a reduced level in accord with present invention. In some embodiments, a suitable dosage level for indomethacin is between about 20 mg to about 1000 mg per day. In other embodiments, a suitable dosage level for indomethacin is between about 40 mg to about 400 mg per day. In still other embodiments, a suitable dosage level for indomethacin is between about 50 mg to about 300 mg per day. Indomethacin may be administered up to six times per day. In some embodiments, indomethacin is administered between 1 and 4 times daily.

When administered in combination, either as a single or as a separate pharmaceutical composition the nicotinic agonist and indomethacin are present in a ratio consistent with the desired effect. In some embodiments, the ratio by weight of indomethacin to the nicotinic agonist is about 10:1. In other embodiments, the ratio by weight of indomethacin to the nicotinic agonist is between about 100:1 and about 1:1. In still other embodiments, the ratio by weight of indomethacin to the nicotinic agonist is between about 20:1 and about 5:1.

When administered in combination, either as a single or as a separate pharmaceutical composition the nicotinic agonist and ibuprofen are present in a ratio consistent with the desired effect. In some embodiments the ratio by weight of ibuprofen to the nicotinic agonist is about 120:1. In other embodiments, the ratio by weight of ibuprofen to the nicotinic agonist is between about 1200:1 and about 12:1. In still other embodiments the ratio by weight of ibuprofen to the nicotinic agonist is between about 240:1 and about 60:1.

Ibuprofen can be administered at a dosage level up to conventional dosage levels for such analgesics but in some embodiments is administered at a reduced level in accord with present invention. In some embodiments, a suitable dosage level for ibuprofen is between about 120 mg to about 6000 mg per day. In other embodiments, a suitable dosage level for ibuprofen is between about 240 mg to about 2400 mg per day. In still other embodiments, a suitable dosage level for ibuprofen is between about 300 mg to about 1800 mg per day. Ibuprofen may be administered up to six times per day. In some embodiments, ibuprofen is administered between 1 and 4 times daily.

When administered in combination, either as a single or as a separate pharmaceutical composition the nicotinic agonist and lumiracoxib are present in a ratio consistent with the desired effect. In some embodiments, the ratio by weight of lumiracoxib to the nicotinic agonist is about 40:1. In other embodiments, the ratio by weight of lumiracoxib to the nicotinic agonist is between about 400:1 and about 4:1. In still other embodiments, the ratio by weight of lumiracoxib to the nicotinic agonist is between about 80:1 and about 20:1.

Lumiracoxib can be administered at a dosage level up to conventional dosage levels for such analgesics but in some embodiments is administered at a reduced level in accord with present invention. In some embodiments, a suitable dosage level for lumiracoxib is between about 40 mg to about 2000 mg per day. In other embodiments, a suitable dosage level for lumiracoxib is between about 80 mg to about 800 mg per day. In still other embodiments, a suitable dosage level for lumiracoxib is between about 100 mg to about 600 mg per day. Lumiracoxib may be administered up to six times per day. In some embodiments, lumiracoxib is administered between 1 and 4 times daily.

When administered in combination, either as a single or as a separate pharmaceutical composition the nicotinic agonist and valdecoxib are present in a ratio consistent with the desired effect. In some embodiments, the ratio by weight of valdecoxib to the nicotinic agonist is about 2:1. In other embodiments, the ratio by weight of valdecoxib to the nicotinic agonist is between about 20:1 and about 0.2:1. In still other embodiments, the ratio by weight of valdecoxib to the nicotinic agonist is between about 4:1 and about 1:1.

Valdecoxib can be administered at a dosage level up to conventional dosage levels for such analgesics but in some embodiments is administered at a reduced level in accord with present invention. In some embodiments, a suitable dosage level for valdecoxib is between about 2 mg to about 100 mg per day. In other embodiments, a suitable dosage level for valdecoxib is between about 4 mg to about 40 mg per day. In still other embodiments, a suitable dosage level for valdecoxib is between about 5 mg to about 30 mg per day. Valdecoxib may be administered up to six times per day. In some embodiments, valdecoxib is administered between 1 and 4 times daily.

When administered in combination, either as a single or as a separate pharmaceutical composition the nicotinic agonist and keterolac are present in a ratio consistent with the desired effect. In some embodiments, the ratio by weight of keterolac to the nicotinic agonist is about 6:1. In other embodiments, the ratio by weight of keterolac to the nicotinic agonist is between about 60:1 and about 1:1. In still other embodiments, the ratio by weight of keterolac to the nicotinic agonist is between about 12:1 and about 3:1.

Keterolac can be administered at a dosage level up to conventional dosage levels for such analgesics but in some embodiments is administered at a reduced level in accord with present invention. In some embodiments, a suitable dosage level for keterolac is between about 6 mg to about 300 mg per day. In other embodiments, a suitable dosage level for keterolac is between about 12 mg to about 120 mg per day. In still other embodiments, a suitable dosage level for keterolac is between about 15 mg to about 90 mg per day. Keterolac may be administered up to six times per day. In some embodiments, keterolac is administered between 1 and 4 times daily.

When administered in combination, either as a single or as a separate pharmaceutical composition the nicotinic agonist and suprofen are present in a ratio consistent with the desired effect. In some embodiments, the ratio by weight of suprofen to the nicotinic agonist is about 20:1. In other embodiments, the ratio by weight of suprofen to the nicotinic agonist is between about 200:1 and about 2:1. In still other embodiments, the ratio by weight of suprofen to the nicotinic agonist is between about 40:1 and about 10:1.

Suprofen can be administered at a dosage level up to conventional dosage levels for such analgesics but in some embodiments is administered at a reduced level in accord with present invention. In some embodiments, a suitable dosage level for suprofen is between about 20 mg to about 1000 mg per day. In other embodiments, a suitable dosage level for suprofen is between about 40 mg to about 400 mg per day. In still other embodiments, a suitable dosage level for suprofen is between about 50 mg to about 300 mg per day. Suprofen may be administered up to six times per day. In some embodiments, suprofen is administered between 1 and 4 times daily.

When administered in combination, either as a single or as a separate pharmaceutical composition the nicotinic agonist and aspirin are present in a ratio consistent with the desired effect. In some embodiments, the ratio by weight of aspirin to the nicotinic agonist is about 130:1. In other embodiments, the ratio by weight of aspirin to the nicotinic agonist is between about 1300:1 and about 13:1. In still other embodiments, the ratio by weight of aspirin to the nicotinic agonist is between about 260:1 and about 65:1.

Aspirin can be administered at a dosage level up to conventional dosage levels for such analgesics but in some embodiments is administered at a reduced level in accord with present invention. In some embodiments, a suitable dosage level for aspirin is between about 130 mg to about 6500 mg per day. In other embodiments, a suitable dosage level for aspirin is between about 260 mg to about 2600 mg per day. In still other embodiments, a suitable dosage level for aspirin is between about 325 mg to about 1950 mg per day. Aspirin may be administered up to six times per day. In some embodiments, aspirin is administered between 1 and 4 times daily.

When administered in combination, either as a single or as a separate pharmaceutical composition the nicotinic agonist and etoricoxib are present in a ratio consistent with the desired effect. In some embodiments, the ratio by weight of etoricoxib to the nicotinic agonist is about 12:1. In other embodiments, the ratio by weight of etoricoxib to the nicotinic agonist is between about 120:1 and about 1:1. In still other embodiments, the ratio by weight of etoricoxib to the nicotinic agonist is between about 24:1 and about 6:1.

Etoricoxib can be administered at a dosage level up to conventional dosage levels for such analgesics but in some embodiments is administered at a reduced level in accord with present invention. In some embodiments, a suitable dosage level for etoricoxib is between about 12 mg to about 600 mg per day. In other embodiments, a suitable dosage level for etoricoxib is between about 24 mg to about 240 mg per day. In still other embodiments, a suitable dosage level for etoricoxib is between about 30 mg to about 180 mg per day. Etoricoxib may be administered up to six times per day. In some embodiments, etoricoxib is administered between 1 and 4 times daily.

When administered in combination, either as a single or as a separate pharmaceutical composition the nicotinic agonist and nimesulide are present in a ratio consistent with the desired effect. In some embodiments, the ratio by weight of nimesulide to the nicotinic agonist is about 30:1. In other embodiments, the ratio by weight of nimesulide to the nicotinic agonist is between about 300:1 and about 3:1. In still other embodiments, the ratio by weight of nimesulide to the nicotinic agonist is between about 60:1 and about 15:1.

Nimesulide can be administered at a dosage level up to conventional dosage levels for such analgesics but in some embodiments is administered at a reduced level in accord with present invention. In some embodiments, a suitable dosage level for nimesulide is between about 30 mg to about 1500 mg per day. In other embodiments, a suitable dosage level for nimesulide is between about 60 mg to about 600 mg per day. In still other embodiments, a suitable dosage level for nimesulide is between about 75 mg to about 450 mg per day. Nimesulide may be administered up to six times per day. In some embodiments, nimesulide is administered between 1 and 4 times daily.

When administered in combination, either as a single or as a separate pharmaceutical composition the nicotinic agonist and meloxicam are present in a ratio consistent with the desired effect. In some embodiments, the ratio by weight of meloxicam to the nicotinic agonist is about 2:1. In other embodiments, the ratio by weight of meloxicam to the nicotinic agonist is between about 15:1 and about 0.2:1. In still other embodiments, the ratio by weight of meloxicam to the nicotinic agonist is between about 3:1 and about 0.8:1.

Meloxicam can be administered at a dosage level up to conventional dosage levels for such analgesics but in some embodiments is administered at a reduced level in accord with present invention. In some embodiments, a suitable dosage level for meloxicam is between about 2 mg to about 75 mg per day. In other embodiments, a suitable dosage level for meloxicam is between about 3 mg to about 30 mg per day. In still other embodiments, a suitable dosage level for meloxicam is between about 4 mg to about 23 mg per day. Meloxicam may be administered up to six times per day. In some embodiments, meloxicam is administered between 1 and 4 times daily.

When administered in combination, either as a single or as a separate pharmaceutical composition the nicotinic agonist and sulindac are present in a ratio consistent with the desired effect. In some embodiments, the ratio by weight of sulindac to the nicotinic agonist is about 30:1. In other embodiments, the ratio by weight of sulindac to the nicotinic agonist is between about 300:1 and about 3:1. In still other embodiments, the ratio by weight of sulindac to the nicotinic agonist is between about 60:1 and about 15:1.

Sulindac can be administered at a dosage level up to conventional dosage levels for such analgesics but in some embodiments is administered at a reduced level in accord with present invention. In some embodiments, a suitable dosage level for sulindac is between about 30 mg to about 1500 mg per day. In other embodiments, a suitable dosage level for sulindac is between about 60 mg to about 600 mg per day. In still other embodiments, a suitable dosage level for sulindac is between about 75 mg to about 450 mg per day. Sulindac may be administered up to six times per day. In some embodiments, sulindac is administered between 1 and 4 times daily.

When administered in combination, either as a single or as a separate pharmaceutical composition the nicotinic agonist and piroxicam are present in a ratio consistent with the desired effect. In some embodiments, the ratio by weight of piroxicam to the nicotinic agonist is about 2:1. In other embodiments, the ratio by weight of piroxicam to the nicotinic agonist is between about 20:1 and about 0.2:1. In still other embodiments, the ratio by weight of piroxicam to the nicotinic agonist is between about 4:1 and about 1:1.

Piroxicam can be administered at a dosage level up to conventional dosage levels for such analgesics but in some embodiments is administered at a reduced level in accord with present invention. In some embodiments, a suitable dosage level for piroxicam is between about 2 mg to about 100 mg per day. In other embodiments, a suitable dosage level for piroxicam is between about 4 mg to about 40 mg per day. In still other embodiments, a suitable dosage level for piroxicam is between about 5 mg to about 30 mg per day. Piroxicam may be administered up to six times per day. In some embodiments, piroxicam is administered between 1 and 4 times daily.

When administered in combination, either as a single or as a separate pharmaceutical composition the nicotinic agonist and bromfenac are present in a ratio consistent with the desired effect. In some embodiments, the ratio by weight of bromfenac to the nicotinic agonist is about 10:1. In other embodiments, the ratio by weight of bromfenac to the nicotinic agonist is between about 100:1 and about 1:1. In still other embodiments, the ratio by weight of bromfenac to the nicotinic agonist is between about 20:1 and about 5:1.

Bromfenac can be administered at a dosage level up to conventional dosage levels for such analgesics but in some embodiments is administered at a reduced level in accord with present invention. In some embodiments, a suitable dosage level for bromfenac is between about 10 mg to about 500 mg per day. In other embodiments, a suitable dosage level for bromfenac is between about 20 mg to about 200 mg per day. In still other embodiments, a suitable dosage level for bromfenac is between about 25 mg to about 150 mg per day. Bromfenac may be administered up to six times per day. In some embodiments, bromfenac is administered between 1 and 4 times daily.

When administered in combination, either as a single or as a separate pharmaceutical composition the nicotinic agonist and biphenylacetic acid are present in a ratio consistent with the desired effect. In some embodiments, the ratio by weight of biphenylacetic acid to the nicotinic agonist is about 2:1. In other embodiments, the ratio by weight of biphenylacetic acid to the nicotinic agonist is between about 20:1 and about 0.2:1. In still other embodiments, the ratio by weight of biphenylacetic acid to the nicotinic agonist is between about 4:1 and about 1:1.

Biphenylacetic acid can be administered at a dosage level up to conventional dosage levels for such analgesics but in some embodiments is administered at a reduced level in accord with present invention. In some embodiments, a suitable dosage level for biphenylacetic acid is between about 2 mg to about 100 mg per day. In other embodiments, a suitable dosage level for biphenylacetic acid is between about 4 mg to about 40 mg per day. In still other embodiments, a suitable dosage level for biphenylacetic acid is between about 5 mg to about 30 mg per day. Biphenylacetic acid may be administered up to six times per day. In some embodiments, biphenylacetic acid is administered between 1 and 4 times daily.

When administered in combination, either as a single or as a separate pharmaceutical composition the nicotinic agonist and rofecoxib are present in a ratio consistent with the desired effect. In some embodiments, the ratio by weight of rofecoxib to the nicotinic agonist is about 5:1. In other embodiments, the ratio by weight of rofecoxib to the nicotinic agonist is between about 50:1 and about 1:1. In still other embodiments, the ratio by weight of rofecoxib to the nicotinic agonist is between about 10:1 and about 3:1.

Rofecoxib can be administered at a dosage level up to conventional dosage levels for such analgesics but in some embodiments is administered at a reduced level in accord with present invention. In some embodiments, a suitable dosage level for rofecoxib is between about 5 mg to about 250 mg per day. In other embodiments, a suitable dosage level for rofecoxib is between about 10 mg to about 100 mg per day. In still other embodiments, a suitable dosage level for rofecoxib is between about 13 mg to about 75 mg per day. Rofecoxib may be administered up to six times per day. In some embodiments, rofecoxib is administered between 1 and 4 times daily.

When administered in combination, either as a single or as a separate pharmaceutical composition the nicotinic agonist and diclofenac potassium are present in a ratio consistent with the desired effect. In some embodiments, the ratio by weight of diclofenac potassium to the nicotinic agonist is about 20:1. In other embodiments, the ratio by weight of diclofenac potassium to the nicotinic agonist is between about 200:1 and about 2:1. In still other embodiments, the ratio by weight of diclofenac potassium to the nicotinic agonist is between about 40:1 and about 10:1.

Diclofenac potassium can be administered at a dosage level up to conventional dosage levels for such analgesics but in some embodiments is administered at a reduced level in accord with present invention. In some embodiments, a suitable dosage level for diclofenac potassium is between about 20 mg to about 1000 mg per day. In other embodiments, a suitable dosage level for diclofenac potassium is between about 40 mg to about 400 mg per day. In still other embodiments, a suitable dosage level for diclofenac potassium is between about 50 mg to about 300 mg per day. Diclofenac potassium may be administered up to six times per day. In some embodiments, diclofenac potassium is administered between 1 and 4 times daily.

### 4.6 Combination Therapy

In certain embodiments, nicotinic agonists and therapeutic agents and/or pharmaceutical compositions thereof can be used in combination therapy with at least one other therapeutic agent. The nicotinic agonists and therapeutic agents and/or pharmaceutical compositions thereof and the therapeutic agent can act additively or, more preferably, synergistically. In some embodiments, nicotinic agonists and therapeutic agents and/or pharmaceutical compositions thereof are administered concurrently with the administration of another therapeutic agent. For example, nicotinic agonists and therapeutic agents and/or pharmaceutical compositions thereof may be administered together with another pain reducing agent or anti-emetic agent. In other embodiments, nicotinic agonists and therapeutic agents and/or pharmaceutical composition thereof are administered prior or subsequent to administration of other therapeutic agents.

### 4.7 Therapeutic Kits

Also provided herein are therapeutic kits comprising the compositions and/or compounds disclosed herein. The therapeutic kits may also contain other therapeutic agents or pharmaceutical compositions of these other agents.

Therapeutic kits may have a single container which contains the compositions and/or compounds or pharmaceutical compositions thereof with or without other components (e.g., other therapeutic agents or pharmaceutical compositions thereof) or may have distinct container for each component. The components of the kit may be pre-complexed or each component may be in a separate distinct container prior to administration to a patient.

The components of the kit may be provided in one or more liquid solutions, preferably, an aqueous solution, more preferably, a sterile aqueous solution. The components of the kit may also be provided as solids, which may be converted into liquids by addition of suitable solvents, which are preferably provided in another distinct container.

The container of a therapeutic kit may be a vial, test tube, flask, bottle, syringe, or any other means of enclosing a solid or liquid. Usually, when there is more than one component, the kit will contain a second vial or other container, which allows for separate dosing. The kit may also contain another container for a pharmaceutically acceptable liquid.

In some embodiments, a therapeutic kit will contain apparatus (e.g., one or more needles, syringes, eye droppers, pipette, etc.), which enables administration of the components of the kit.

It will be apparent to those skilled in the art that many modifications, both to materials and methods, may be practiced without departing from the scope of this disclosure. Accordingly, the present embodiments are to be considered as illustrative and not restrictive, and the invention is not to be limited to the details given herein, but may be modified within the scope and equivalents of the allowed claims.

All publications and patents cited herein are incorporated by reference in their entirety.

What is claimed is:

1. A composition comprising a therapeutically effective amount of a nicotinic agonist or salts, hydrates, solvates or prodrugs thereof and a therapeutically effective amount of a therapeutic agent selected from the group consisting of non-steroidal anti-inflammatory drugs, COX1 inhibitors and COX2 inhibitors.

2. A pharmaceutical composition comprising the composition of claim 1 and a pharmaceutically acceptable vehicle.

3. The composition of any one of claims 1-2 in which the nicotinic agonist is nicotine, meta-nicotine, DMPP, DMAC, ABT 148, DMBX-anabaseine, choline, acetylcholine, epibatidine, cytisine, GTS-21 or an alpha selective nicotinic agonist.

4. The composition of any one of claims 1-2 in which the nicotinic agonist is nicotine.

5. The composition of any one of claims 1-2 in which the therapeutic agent is non-steroidal anti-inflammatory drug selected from the group consisting of aspirin, biphenylacetic acid, acetaminophen, diflunisal, choline magnesium trisalicylate, indomethacin, ibuprofen, ketoprofen, fenoprofen, oxaprozin, bromfenac, felbinac, piroxicam, naproxen, naproxen sodium, diclofenac potassium, mefenamic acid, meloxicam, piroxicam, nabumetone, nimesulfide, etodolac, keterolac, tolmetin, lysine acetylsalicylate, suprofen, suldinac, celecoxib, valdecoxib, parecoxib, refecoxib, rofecoxib, etoricoxib or lumiracoxib.

6. The composition of claim 5 in which the non-steroidal anti-inflammatory drug is a COX-2 enzyme inhibitor.

7. The composition of claim 6 in which the COX-2 enzyme inhibitor is celecoxib, valdecoxib, parecoxib, rofecoxib, etoricoxib or lumiracoxib.

8. The composition of any one of claims 1-2 in which the nicotinic agonist is nicotine and the non-steroidal anti-inflammatory drug is aspirin, biphenylacetic acid, acetaminophen, diflunisal, choline magnesium trisalicylate, indomethacin, ibuprofen, ketoprofen, fenoprofen, oxaprozin, bromfenac, felbinac, piroxicam, naproxen, naproxen sodium, diclofenac potassium, mefenamic acid, meloxicam, piroxicam, nabumetone, nimesulfide, etodolac, keterolac, tolmetin, lysine, acetylsalicylate, suprofen, suldinac, celecoxib, valdecoxib, parecoxib, refecoxib, rofecoxib, etoricoxib or lumiracoxib.

9. The composition of any one of claims 1-2 in which the nicotinic agonist is nicotine and the non-steroidal anti-inflammatory drug is a COX-2 enzyme inhibitor.

10. The composition of claim 9 in which the COX-2 enzyme inhibitor is celecoxib, valdecoxib, parecoxib, rofecoxib, etoricoxib or lumiracoxib.

11. A method of treating or preventing disease or pain or migraine in a subject comprising administering to the subject in need of such treatment or prevention the composition of any one of claims 1-2.

12. A method of treating chronic pain in a subject comprising administering to the subject in need of such treatment a therapeutically effective amount of the composition of any one of claims 1-2.

13. A method of treating or preventing acute pain in a subject comprising administering to the subject in need of such treatment the composition of any one of claims 1-2.

14. A method of treating or preventing disease or pain in a subject comprising administering to the subject in need of such treatment or prevention the composition of any one of claims 1 or 2 wherein the relief to the subject is substantially similar or greater than the relief provided by administration of the therapeutically effective amount of the therapeutic agent or salts, hydrates, solvates or prodrugs thereof or the therapeutically effective amount of the nicotinic agonist or salts, hydrates, solvates or prodrugs thereof.

15. The composition of any one of claims 1-2 comprising a sustained release dosage form.

16. The composition of claim 15 in which the sustained release dosage form is an oral dosage form.

17. The composition of claim 15 in which the sustained release dosage form is a transdermal patch.

18. The composition of any one of claims 1-2 in which the nicotinic agonist is released over the therapeutically effective lifetime of the therapeutic agent.
